(19) United States
(12) Patent Application Publication
(10) Pub. No.: US 2019/0323969 A1
(43) Pub. Date: Oct. 24, 2019

(54) ZINC TRANSPORTER 8 ANTIBODY CHEMILUMINESCENCE IMMUNOASSAY KIT AND PREPARATION METHOD THEREOF

(71) Applicant: SHENZHEN YHLO BIOTECH CO., LTD., Shenzhen (CN)

(72) Inventors: Lin HE, Shenzhen (CN); Qiaohong CHEN, Shenzhen (CN); Yonghong YANG, Shenzhen (CN); Fuzhen XIA, Shenzhen (CN); Chungen QIAN, Shenzhen (CN); Xing LIU, Shenzhen (CN)

(73) Assignee: SHENZHEN YHLO BIOTECH CO., LTD., Shenzhen (CN)

(21) Appl. No.: 16/313,905

(22) PCT Filed: Apr. 13, 2017
(86) PCT No.: PCT/CN2017/080402

§ 371 Date: Dec. 28, 2018

(30) Foreign Application Priority Data
Jun. 30, 2016 (CN) 201610510235.4

(51) Int. Cl. G01N 21/76; G01N 33/532; G01N 33/553

(52) U.S. Cl. CPC G01N 21/76; G01N 33/553; G01N 33/532

(57) ABSTRACT

The present invention discloses a zinc transporter 8 antibody chemiluminescence immunoassay kit, including a magnetic microparticle working solution coated with purified zinc transporter 8, a purified zinc transporter 8 working solution labeled with acridinium ester, a zinc transporter 8 antibody calibrator, a chemiluminescent pre-excitation solution and a chemiluminescent excitation solution. In addition, the present invention also discloses a method for preparing the zinc transporter 8 antibody chemiluminescence immunoassay kit.

### CROSS REFERENCE TO RELATED APPLICATIONS

This application is the national phase entry of International Application No. PCT/CN2017/080402, filed on Apr. 13, 2017, which is based upon and claims priority to Chinese Patent Application No. 201610510235.4, filed on Jun. 30, 2016, the entire contents of which are incorporated herein by reference.

### TECHNICAL FIELD

The present invention relates to the field of diagnostic immunoassay in vitro, and in particular, the present invention provides a zinc transporter 8 antibody chemiluminescence immunoassay kit and a preparation method thereof.

### BACKGROUND

Zinc transporter 8 (ZnT8) is mainly located in islet β cells, and can transport cytosolic zinc ions into insulin storage/secretory vesicles. The decrease of such a transport function will affect insulin synthesis, storage and secretion, and can increase the risk of type 1 diabetes mellitus (T1DM). ZnT8 protein can also cause autoimmune damage of β cells and induce type 1 diabetes mellitus (T1DM).

The expression of ZnT8 protein can increase the storage of vesicle zinc and total content of cytosolic zinc and promote insulin secretion when blood glucose is elevated. Therefore, stimulation of ZnT8 protein to increase its synthesis or enhance function is expected to reduce the damage of hypozincemia to diabetic patients, prevent the apoptosis of β cells resulting from intracellular zinc depletion and/or oxidative stress injury induced by the apoptosis. In addition, ZnT8 protein is immunogenic, and can cause autoimmune injury of β cells as an antigen. Related antibodies developed for ZnT8 protein are important for the prevention or treatment of T1DM. Common methods for clinical detection of ZnT8 antibodies are radioimmunoassay and enzyme-linked immunosorbent assay, but these methods have some shortcomings.

1. Radioimmunoassay

The basic principle of the method is that a recombinant ZnT8 antigen is firstly labeled with radioactive I¹²⁵, an antigen-antibody complex is formed by binding the specific antibody in serum with the antigen, a secondary antibody is added, the mixture is incubated and then an antigen-antibody-secondary antibody complex is formed, and after centrifugation, the radioactivity is measured to determine the specific antibody content in the serum. The method is relatively mature in technology, but its shortcomings are obvious:

(1) Radioactivity affects an operator's body;

(2) The operation is relatively complicated, requiring a centrifuge and an emissive detection device, which are difficult for many primary hospitals to promote;

(3) The background is high and the specificity is not good.

2. Enzyme-Linked Immunosorbent Assay

Enzyme-linked immunosorbent assay (ELISA) is widely used, but this method also has the following shortcomings:

(1) A 12×8 type, 6×8 type, 8×12 type or full-plate type 96-well dedicated microplate is used as an antigen-coating tool and a reaction container, which can only be divided into 12 batches, 6 batches, 8 batches or 1 batch in a single plate during use, and an independent and individual assay cannot be performed;

(2) There are many kinds of reagents used for quantitative determination. Each test reagent should be placed in a reagent vial, and a pipette tip needs to be replaced to be separately added to wells of the microplate when using each reagent. Not only are the types of reagent vials numerous, but also the operation of adding the reagents is extremely cumbersome;

(3) The corresponding labeling of detection information is lacking, the production batch number and expiration date of the detection reagent can only be understood or known by checking a label on an outer packing box of the kit, and the known information is not controlled during a detection process, and has great randomness;

(4) The detection reagent is in an open space during the detection process, which easily causes cross-contamination between various reagents and thus affects the accuracy of detection results;

(5) The detection process is mostly manually operated, the additive amount of reagents or samples is not very precise, the operation process is extremely cumbersome and complicated, which is prone to operational errors, and the accuracy and precision of the detection results are poor.

### SUMMARY

At present, the detection technology for a ZnT8 antibody has the following disadvantages: high detection cost, low detection sensitivity, narrow detection linear range, low reproducibility, inability to quantify, and complicated operation, etc.

In order to overcome the above disadvantages, the present invention discloses a ZnT8 antibody kit with low detection costs, high sensitivity, a wide detection linear range, high reproducibility, and a quantitative and simple operation, and a preparation method thereof. According to the present invention, firstly, a chemiluminescence immunoassay kit is prepared, which mainly includes: a magnetic microparticle coated with zinc transporter member 8, an acridinium ester coated with the ZnT8 and a ZnT8 antibody calibrator; the calibrator is tested using a fully automatic chemiluminescence immunoassay analyzer, a standard curve is plotted and internally installed into computer software, an actual sample is tested, and the sample concentration is calculated according to a sample luminescence value; finally, the performance (sensitivity, linearity, precision, and interference) of a fully automatic chemiluminescence immunoassay system for the ZnT8 antibody is evaluated.

A ZnT8 antibody chemiluminescence immunoassay kit is provided. The kit includes: a solid phase carrier working solution coated with purified zinc transporter member 8, a purified ZnT8 working solution labeled with an acridinium ester, a ZnT8 antibody calibrator and a chemiluminescent substrate solution.

The solid phase carrier is a magnetic microparticle.

The magnetic microparticle is a carboxylated magnetic microparticle having a particle diameter of 0.05 to 1 um.

The chemiluminescent label is an acridinium ester, an acridinium sulfonamide ester, an acridinium toluenesulfonamide ester, an acridinium p-methylsulfonamide ester or an acridinium trifluoromethylsulfonamide ester.

The chemiluminescent label is an acridinium ester.

The chemiluminescent substrate solution includes a chemiluminescent excitation solution and a chemiluminescent pre-excitation solution.

The chemiluminescent pre-excitation solution is a hydrogen peroxide solution with a mass fraction of 0.005% to 0.5%, and the chemiluminescence excitation solution is a 0.005 mol/L to 0.025 mol/L of sodium hydroxide solution.

The ZnT8 antibody calibrator is obtained by formulating the ZnT8 antibody to a solution with a concentration of 5 AU/mL, 20 AU/mL, 80 AU/mL, 200 AU/mL, 800 AU/mL, and 2000 AU/mL using a standard buffer, sub-packaging, lyophilizing and storing at 4° C. for use.

A method for preparing the kit includes the preparation of the magnetic microparticle coated with zinc transporter member 8, preparation of the acridinium ester labeled with the zinc transporter member 8, preparation of the chemiluminescent substrate solution, and preparation of the ZnT8 antibody calibrator.

The method for preparing the kit includes the following steps:

1) preparation of the magnetic microparticle coated with the zinc transporter member 8:

magnetically separating carboxylated nano-magnetic bead suspension, removing supernatant, resuspending with an MES buffer, adding an EDC aqueous solution to activate the carboxyl group on a surface of a magnetic bead, adding the zinc transporter member 8, suspending for 2 to 10 h at room temperature, magnetically separating, removing the supernatant, and resuspending with a Tris buffer to obtain the magnetic microparticle coated with the zinc transporter member 8, where the carboxylated nano-magnetic bead has a diameter of 0.1 μm to 2.0 μm, the MES buffer has a concentration of 10 mM to 100 mM, and pH is 5.5 to 8.5;

2) preparation of the ZnT8 label labeled with the acridinium ester:

taking zinc transporter member 8, adding into a carbonate buffer, mixing uniformly, then adding the acridinium ester, mixing uniformly, reacting in the dark at room temperature, taking out after 1 to 2 h, desalting by a centrifugal desalting column, during desalting deionized water and a TBS buffer being used for treating, respectively, then adding an obtained acridinium ester solution labeled with the zinc transporter member 8, and collecting liquid in a centrifuge tube into a preservation tube to obtain the acridinium ester labeled with the zinc transporter member 8;

3) preparation of the ZnT8 antibody calibrator:

formulating an ZnT8 antibody to a concentration of 5 AU/mL, 20 AU/mL, 80 AU/mL, 200 AU/mL, 800 AU/mL and 2000 AU/mL using a standard buffer, subpackaging, lyophilizing and storing at 4° C. for use;

4) preparation of the chemiluminescent pre-excitation solution:

measuring 1.0 L of purified water, adding 0.5 to 100 μL of hydrogen peroxide (H₂O₂) with a mass fraction of 20%, 0.5 to 5 g of preservative, and 0.5 to 5 g of surfactant in sequence, shaking uniformly, and then storing in the dark, where the preservative is commercially available sodium azide or PC300, and the surfactant is Tween 20, Tween 80, Triton X-100, or Triton X-405; and

5) preparation of the chemiluminescent excitation solution:

measuring 1.0 L of purified water, adding 0.2-1 g of sodium hydroxide, 0.5 to 5 g of preservative, and 0.5 to 5 g of surfactant in sequence, shaking uniformly, and then storing in the dark, where the preservative is commercially available sodium azide or PC300, and the surfactant is Tween 20, Tween 80, Triton X-100, or Triton X-405.

The present invention has the following advantages in comparison with the prior art:

1. The present invention selects an acridinium ester as a labeling material and applies it to a chemiluminescence immunoassay system that is direct chemiluminescence, and compared with conventional enzymatic chemiluminescence, such reaction does not require the participation of an enzyme and is more cost effective;

2. The acridinium ester chemiluminescence immunoassay system selected in the present invention has high detection sensitivity up to 0.06 AU/mL, which is increased by at least 12 times than other ZnT8 antibody detection methods in sensitivity;

3. The acridinium ester chemiluminescence immunoassay system used in the present invention has a wide linear range up to 10 to 2000 AU/mL, while the linear range of other ZnT8 antibody detection methods is 20 to 1000 AU/mL;

4. The acridinium ester chemiluminescence immunoassay system used in the present invention has high repeatability, and differences within a batch and between batches are both within 5%, which is difficult to achieve for other chemiluminescence immunoassay systems;

5. The chemiluminescence immunoassay system of the present invention has realized the sample quantitative detection, and upon internally setting a standard curve to test software, a sample concentration value can be directly obtained by testing the sample;

6. The chemiluminescence immunoassay system of the present invention has realized the complete automation, the addition of reagents and samples are both performed by instruments, which results in a simpler operation and reduced human error.

### BRIEF DESCRIPTION OF THE DRAWINGS

FIG. 1: a diagram showing a ZnT8 antibody standard curve.

### DETAILED DESCRIPTION

Embodiment 1: Method for preparing a ZnT8 antibody chemiluminescence immunoassay kit

(1) Preparation of a nano-magnetic bead coated with zinc transporter member 8:

50 mg of carboxylated magnetic particle (particle size 0.05 to 1 um) suspension was taken, and magnetically separated, and a supernatant was removed. Resuspending was performed with 0.02 M of MES buffer at pH 5.5, and 0.5 to 2 mL of fresh prepared 10 mg/mL EDC aqueous solution was added to activate carboxyl group on a surface of a magnetic bead. 3 to 5 mg of glutamic acid was added for decarboxylation. The mixture was suspended for 2 to 10 h at room temperature, and magnetically separated. A supernatant was removed, and resuspending to 1 mg/mL was performed with 0.1 M of Tris buffer containing 2% BSA and at pH 8.0 to obtain the magnetic microparticle coated with a ZnT8 monoclonal antibody, which was packed in vials (5 mL per vial) and stored at 4° C. for use.

(2) Preparation of a ZnT8 label labeled with an acridinium ester:

50 μL of 25 mg/mL ZnT8 was taken, and added into 150 μL of 0.1 to 0.2 M carbonate buffer at pH 9.0 to 9.5 to be mixed uniformly. After that, 1 to 2 μL of 5 mg/mL acridinium ester was added to be mixed uniformly and reacted in the dark at room temperature. A product was taken out after 1 to 2 h, and desalted by 2 mL of zeba spin desalting column, during desalting, treating was performed with deionized water and a TBS buffer, respectively. Then an obtained acridinium ester solution labeled with ZnT8 was added. Liquid in a centrifuge tube was collected into a preservation tube to obtain the acridinium ester labeled with the zinc transporter member 8, which was packed in vials (5 mL per vial) and stored at 4° C. for use.

(3) Preparation of a ZnT8 antibody calibrator:

The ZnT8 antibody was formulated to a certain concentration of 5 AU/mL, 20 AU/mL, 80 AU/mL, 200 AU/mL, 800 AU/mL and 2000 AU/mL using a standard buffer (40 mM Tris-HCl, 0.5% BSA, 1% NaCl, pH 8.0), which was packed in vials (0.5 mL per vial), lyophilized and stored at 4° C. for use.

(4) Preparation of a chemiluminescent pre-excitation solution:

1.0 L of deionized water was measured, and 80 μL of hydrogen peroxide (H₂O₂) with a mass fraction of 20%, 1.0 g of sodium azide, and 1.5 g of Tween 20 were added in sequence. Shaking uniformly and then storing in the dark were performed.

(5) Preparation of a chemiluminescent excitation solution:

1.0 L of deionized water was measured, and 0.6 g of sodium hydroxide, 0.5 g of PC300, 0.5 g of sodium azide, and 1.5 g of Triton 405 were added in sequence. Shaking uniformly and then storing in the dark were performed.

Embodiment 2: Chemiluminescence immunoassay of a ZnT8 antibody:

The present invention adopted a fully automatic chemiluminescence immunoassay analyzer as a detection tool, and the method model of the present invention was a double antigen sandwich method. Namely 25 μL of sample, 50 μL of magnetic microparticle coated with the ZnT8 and 50 μL of acridinium ester labeled with the ZnT8 were added to the instrument in sequence, and after reaction for 10 min, magnetic separation was performed. A reaction mixture was fed into a dark room by the instrument. 50 μL of chemiluminescent pre-excitation solution and 50 μL of chemiluminescent excitation solution were added in sequence for luminescence reaction, and finally luminous intensity was recorded. The ZnT8 antibody content of the tested sample was calculated from a standard curve.

Embodiment 3: Performance evaluation of a ZnT8 antibody chemiluminescence immunoassay kit

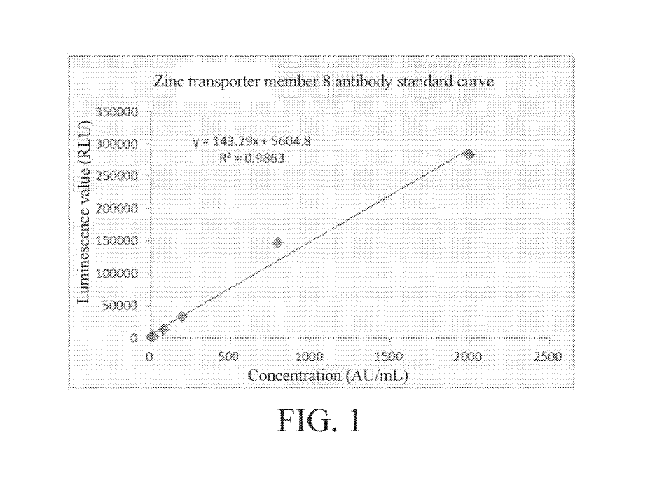

A test curve is shown in FIG. 1

Sensitivity detection:

In reference to an experimental protocol recommended by CLSI EP17-A document, the sensitivity of the ZnT8 antibody chemiluminescence immunoassay kit was calculated, and the obtained sensitivity was 0.06 IU/mL.

Linear detection:

Linear analysis was performed on standards at concentrations of 5 AU/mL, 20 AU/mL, 80 AU/mL, 200 AU/mL, 800 AU/mL and 2000 AU/mL, and a linear correlation coefficient was calculated, r=0.9863. In addition, the kit had a linear range for ZnT8 antibody sample detection of 5 to 2000 AU/mL.

Precision determination:

Two ZnT8 antibody samples at concentrations of 20 AU/mL and 800 AU/mL were taken, each sample at each concentration was determined in triplicate, and the test was performed in three batches of kits. Differences within a batch and between batches for the kit were calculated, and results showed that the differences within a batch and between batches for the kit were both less than 5%.

Interference experiments:

Mixed serum was taken, and interfering substances including bilirubin, hemoglobin, ascorbic acid and glyceride in an addition ratio of 1:20 were added respectively. Measurement values of the mixed serum and the mixed serum with various added interfering substances were respectively measured, and a difference therebetween was calculated with an acceptable range of ±10%. Results showed that the interference reached the standard in the NCCLS document, and usability for accurate evaluation of the ZnT8 antibody in clinical laboratories was achieved.

Embodiment 4: Comparison experiment for analytical sensitivity of a ZnT8 antibody chemiluminescence immunoassay kit

Sample diluent at a concentration of 0 IU/mL were detected by the chemiluminescence detection method and conventional enzyme-linked immunosorbent assay, respectively. The test was repeated 20 times, and RLU (relative light unit) values of 20 measurement results were obtained, and a mean (M) and standard deviation (SD) were calculated to obtain M+2SD. These luminescence values were substituted into a calibration curve to calculate corresponding concentration values. The concentration value obtained by the chemiluminescence detection method was 0.06 IU/mL, 12 times more sensitive than the concentration value obtained was 0.71 IU/mL by the conventional enzyme-linked immunosorbent assay.

What is claimed is:

1. A zinc transporter 8 antibody chemiluminescence immunoassay kit, comprising a solid phase carrier working solution coated with purified zinc transporter 8, a purified zinc transporter 8 working solution labeled with an acridinium ester, a zinc transporter 8 antibody calibrator and a chemiluminescent substrate solution.

2. The zinc transporter 8 antibody chemiluminescence immunoassay kit according to claim 1, wherein a solid phase carrier of the solid phase carrier working solution is a magnetic microparticle.

3. The zinc transporter 8 antibody chemiluminescence immunoassay kit according to claim 2, wherein the magnetic microparticle is a carboxylated magnetic microparticle with a particle diameter of 0.05 to 1 μm.

4. The zinc transporter 8 antibody chemiluminescence immunoassay kit according to claim 1, wherein a chemiluminescent label of the chemiluminescent substrate solution is an acridinium ester, an acridinium sulfonamide ester, an acridinium toluenesulfonamide ester, an acridinium p-methylsulfonamide ester or an acridinium trifluoromethylsulfonamide ester.

5. The zinc transporter 8 antibody chemiluminescence immunoassay kit according to claim 1, wherein a chemiluminescent label of the chemiluminescent substrate solution is an acridinium ester.

6. The zinc transporter 8 antibody chemiluminescence immunoassay kit according to claim 1, wherein the chemiluminescent substrate solution comprises a chemiluminescent excitation solution and a chemiluminescent pre-excitation solution.

7. The zinc transporter 8 antibody chemiluminescence immunoassay kit according to claim 6, wherein the chemiluminescent pre-excitation solution is a hydrogen peroxide solution with a mass fraction of 0.005% to 0.5%, and the chemiluminescence excitation solution is a 0.005 mol/L to 0.025 mol/L of sodium hydroxide solution.

8. The zinc transporter 8 antibody chemiluminescence immunoassay kit according to claim 1, wherein the zinc transporter 8 antibody calibrator is obtained by formulating zinc transporter 8 antibody to solutions respectively with a concentration of 5 AU/mL, 20 AU/mL, 80 AU/mL, 200 AU/mL, 800 AU/mL and 2000 AU/mL using a standard buffer, sub-packaging, lyophilizing and storing at 4° C. for use.

9. A method for preparing a zinc transporter 8 antibody chemiluminescence immunoassay kit, wherein
the zinc transporter 8 antibody chemiluminescence immunoassay kit comprises a solid phase carrier working solution coated with purified zinc transporter 8, a purified zinc transporter working solution labeled with an acridinium ester, a zinc transporter 8 antibody calibrator and a chemiluminescent substrate solution; and wherein the solid phase carrier working solution is a magnetic microparticle working solution coated with purified zinc transporter 8, and the chemiluminescent substrate solution comprises a chemiluminescent excitation solution and a chemiluminescent pre-excitation solution; and wherein
the method comprises preparation of the magnetic microparticle working solution coated with the purified zinc transporter 8, preparation of the purified zinc transporter 8 working solution labeled with the acridinium ester, preparation of the chemiluminescent substrate solution, and preparation of the zinc transporter 8 antibody calibrator.

10. The method according to claim 9, comprising the following steps:
1) preparation of the magnetic microparticle working solution coated with the purified zinc transporter 8:
magnetically separating a carboxylated nano-magnetic bead suspension to remove supernatant, resuspending with an MES buffer to obtain a magnetic bead solution, adding an EDC aqueous solution to activate carboxyl group on a surface of a magnetic bead of the magnetic bead solution, adding a zinc transporter 8, suspending for 2 to 10 h at room temperature, magnetically separating, removing supernatant, and resuspending with a Tris buffer to obtain the magnetic microparticle working solution coated with the purified zinc transporter 8, wherein a carboxylated nano-magnetic bead of the carboxylated nano-magnetic bead suspension has a diameter of 0.1 μm to 2.0 μm, and the MES buffer has a concentration of 10 mM to 100 mM, and has a pH of 5.5 to 8.5;
2) preparation of the purified zinc transporter 8 working solution labeled with the acridinium ester:
taking a zinc transporter 8, adding into a carbonate buffer, mixing uniformly, then adding the acridinium ester, mixing uniformly, reacting in the dark at room temperature, taking out after 1 to 2 h to obtain a zinc transporter 8 solution labeled with the acridinium ester, desalting by a centrifugal desalting column; during the desalting, deionized water and a TBS buffer being used for treating the centrifugal desalting column, respectively; then adding the obtained zinc transporter 8 solution labeled with the acridinium ester into the centrifugal desalting column, and then collecting liquid in a centrifuge tube into a preservation tube to obtain the purified zinc transporter 8 working solution labeled with the acridinium ester;
3) preparation of the zinc transporter 8 antibody calibrator:
formulating an zinc transporter 8 antibody to zinc transporter 8 antibody calibrators respectively with a concentration of 5 AU/mL, 20 AU/mL, 80 AU/mL, 200 AU/mL, 800 AU/mL and 2000 AU/mL using a standard buffer, subpackaging, lyophilizing and storing at 4° C. for use;
4) preparation of the chemiluminescent pre-excitation solution:
measuring 1.0 L of purified water, adding 0.5 to 100 μL of hydrogen peroxide (H₂O₂) with a mass fraction of 20%, 0.5 to 5 g of preservative, and 0.5 to 5 g of surfactant in sequence, shaking uniformly, and then storing in the dark, wherein the preservative is commercially available sodium azide or PC300 preservative, and the surfactant is Tween 20, Tween 80, Triton X-100, or Triton X-405; and
5) preparation of the chemiluminescent excitation solution:
measuring 1.0 L of purified water, adding 0.2-1 g of sodium hydroxide, 0.5 to 5 g of preservative, and 0.5 to 5 g of surfactant in sequence, shaking uniformly, and then storing in the dark, wherein the preservative is commercially available sodium azide or PC300 preservative, and the surfactant is Tween 20, Tween 80, Triton X-100, or Triton X-405.

11. The method according to claim 9, wherein a magnetic microparticle of the magnetic microparticle working solution is a carboxylated magnetic microparticle with a particle diameter of 0.05 to 1 μm.

12. The method according to claim 9, a chemiluminescent label of the chemiluminescent substrate solution is an acridinium ester, an acridinium sulfonamide ester, an acridinium toluenesulfonamide ester, an acridinium p-methylsulfonamide ester or an acridinium trifluoromethylsulfonamide ester.

13. The method according to claim 9, wherein a chemiluminescent label of the chemiluminescent substrate solution is an acridinium ester.

14. The method according to claim 9, wherein the chemiluminescent pre-excitation solution is a hydrogen peroxide solution with a mass fraction of 0.005% to 0.5%, and the chemiluminescence excitation solution is a 0.005 mol/L to 0.025 mol/L of sodium hydroxide solution.

15. The method according to claim 10, wherein a magnetic microparticle of the magnetic microparticle working solution is a carboxylated magnetic microparticle with a particle diameter of 0.05 to 1 μm.

16. The method according to claim 10, a chemiluminescent label of the chemiluminescent substrate solution is an acridinium ester, an acridinium sulfonamide ester, an acridinium toluenesulfonamide ester, an acridinium p-methylsulfonamide ester or an acridinium trifluoromethylsulfonamide ester.

17. The method according to claim 10, wherein a chemiluminescent label of the chemiluminescent substrate solution is an acridinium ester.

18. The method according to claim 10, wherein the chemiluminescent pre-excitation solution is a hydrogen peroxide solution with a mass fraction of 0.005% to 0.5%, and the chemiluminescence excitation solution is a 0.005 mol/L to 0.025 mol/L of sodium hydroxide solution.
